(19) United States
(12) Patent Application Publication
(10) Pub. No.: US 2014/0288648 A1
(43) Pub. Date: Sep. 25, 2014

(54) NEW INTRA-LARYNGEAL ENDOPROSTHESIS

(71) Applicants: Andre Michel Charles Walder, L'Hayes-les-Roses (FR); Sylvain Lefebvre, Vuillafans (FR); Nicolas Perrin, Schiltigheim (FR); Maurice Berenger, Strasbourg (FR)

(72) Inventors: Andre Michel Charles Walder, L'Hayes-les-Roses (FR); Sylvain Lefebvre, Vuillafans (FR); Nicolas Perrin, Schiltigheim (FR); Maurice Berenger, Strasbourg (FR)

(73) Assignee: Protip SAS, Strasbourg (FR)

(21) Appl. No.: 14/343,321

(22) PCT Filed: Sep. 6, 2012
(86) PCT No.: PCT/FR2012/051996

§ 371 Date: May 13, 2014

(30) Foreign Application Priority Data
Sep. 6, 2011 (FR) FR1102694

(51) Int. Cl. A61F 2/20

(52) U.S. Cl. CPC A61F 2/20
USPC 623/9

(57) ABSTRACT

The present invention relates to the field of prosthetics having the function of restoring swallowing, breathing and phonation to a patient having a dysfunctional larynx. More specifically, the present invention concerns a valve device forming an intra-laryngeal endoprosthesis intended for implanting in the anatomical larynx in place having the function of enabling breathing while forming a seal against elements such as saliva, mucus or any other element coming from the bolus. The complete intra-laryngeal endoprosthesis, as well as its various applications, are also the subject matter of the present invention.

The present invention relates to the field of prostheses intended to restore both swallowing, breathing and speech in patients with a dysfunctional larynx. More specifically, the present invention relates to a flap valve device forming an intralaryngeal prosthesis intended to be implanted within an in-situ anatomical larynx the function of which is to allow breathing while providing a seal against elements such as saliva, mucus, or any other element derived from the alimentary bolus. The complete intralaryngeal prosthesis and its various applications also form part of the subject matter of the present invention.

There are various pathologies or consequences of pathologies and/or of treatments which lead patients to present dysfunctions of the larynx. Some of these events lead to partial or total paralysis, which may be either temporary or permanent. The larynx is an osteo-cartilagenous duct the main role of which is of a respiratory nature. Any dysfunctionality may therefore compromise the breathing function in the patient. The larynx plays an important role in the swallowing process insofar as its closure protects the lower airways. Any dysfunctionality therefore leads to problems of undesirable elements, liquid or solid, entering the lower airways.

The prior art has only very few solutions to offer to remedy a dysfunctional larynx because the larynx has many functions and requires complex prostheses the placement of which involves a surgical procedure. It is therefore necessary for the device to be suited to the various functions of the larynx and also to have a substantial life span under physiological conditions so as to limit the operations that the surgeon has to perform. Moreover, the device needs to be made from a material that is biocompatible, strong, light, durable and has to have a shape best suited to the site at which it will be implanted, in order to cause the least trouble to the patient.

Patent application EP 0815807 describes a prosthesis intended to remedy a dysfunctional larynx, said prosthesis consisting of a silicone tube which at one of its ends shaped into a bevel has, on the one hand, an inclined shut-off face and, on the other hand, lateral orifices for communication between the inside and the outside of the tube that forms the prosthesis. In practice, the inclined shut-off face directs the alimentary bolus from the mouth toward the esophagus, whereas the lateral communication orifices allow air to circulate in both directions. Nevertheless, it is true that leaks may occur through those lateral orifices and, more particularly, fluid or reflux of fluid or any other element of the alimentary bolus may enter the lower airways. This is because in practice, the orientation of the shut-off face is not enough on its own to ensure total sealing.

Patent application EP 08872036 also describes an implant system intended to restore respiratory, speech and swallowing functions in a patient with a dysfunctional larynx. That application describes a flap valve device made at least in part from a semi-rigid, biocompatible lightweight and strong material, for example silicone. The device described is preferably made up of a silicone structure, silicone valve flaps and a peripheral reinforcing ring made of titanium which is embedded in the silicone of the structure. The novelty of that device lies in a double-flaps system the objective of which is to allow breathing and swallowing while forming a perfectly fluidtight shutter (obturator) thanks to a bevel cut at the circumferences of the two circular valve flaps and at the circumference of the internal surface of the annular bearing structure. That device has a life that is limited because of the deformation of the valve flaps under physiological conditions and because mold appears due to the presence of the silicone which is readily colonized by undesirable cells. The device has therefore to be replaced in the relatively short term.

These devices of the prior art have the major defect of being sensitive to wear and to colonization under physiological conditions, notably by mold. Specifically, any deformation of the shutter inevitably leads to a lack of sealing and therefore to poor operation of the device that is supposed to remedy the dysfunctionality of the larynx. It is therefore necessary for a surgeon to operate to replace the defective element or elements in the devices of the prior art.

The present invention seeks to overcome these disadvantages by proposing a new device which, on the one hand, is able to ensure perfect sealing of the device by preventing any leaks to the lower airways and, on the other hand, is able to allow air to circulate in both directions to allow the patient to breathe and to perform the functions of speech and swallowing. In addition, the novelty of this invention which is detailed hereinbelow lies in the fact that the device according to the invention has excellent resistance to wear and to deformation under physiological conditions and exhibits a life that is far longer than the devices of the prior art.

To achieve this, the present invention proposes a novel approach compared with the flap valve device described in the patent application EP 08872036. In order to develop a device which is more resistant and performs better in a physiological environment, the inventors have proposed to machine components from biocompatible metal, which are then combined with one another to form a sealed shutter. The biocompatible metals known to date are titanium or titanium-based alloys, gold or gold-based alloys, and platinum or platinum-based alloys, niobium and tantalum or alloys of these. The device made of a semi-rigid material described in patent application EP 08872036 is easy to manufacture by molding or injection-molding for example, because semi-rigid materials are easy to work and are flexible. Wishing to produce a flap valve device from solid metal that is intended to be implanted within a dysfunctional larynx represents a real technical challenge. This is because the prior art in the field of tracheal implants, whether these are implanted via a tracheotomy or via an endo-buccal route, leans rather toward the use of semi-rigid materials because these can be folded for the purposes of insertion in order to be the least traumatic possible both to the implantation tissues and to the anatomical areas through which they will pass before the device is in place. By contrast, in the field of prostheses or implants that require high mechanical stresses such as bone implants, there is a leaning toward the use of rigid materials, including metallic materials. However, said implants or prostheses are not in motion and are there to stabilize or encourage bone growth. The present invention goes against the prior art in the field of laryngology by developing a flap valve device, which therefore involves moving parts, mainly made out of solid metal.

The subject matter of the invention is particularly innovative because it offers exceptional life span, is particularly good at resisting deformation and is also able to allow air to circulate during inhalation and exhalation phases while at the same time protecting the lower airways by shutting off when needs be; thus the functions of swallowing, breathing and speech are restored in a lasting fashion.

More particularly, the present invention relates in general to a flap valve device forming an intralaryngeal prosthesis made of biocompatible solid metal and intended to be implanted in a dysfunctional larynx, said device comprising a distal portion forming an annular bearing structure and a central portion forming a shutter intended to allow air to pass and to hermetically prevent any other element from passing, said device being characterized in that the shutter comprises i) a peripheral part forming a first valve flap secured to the annular bearing structure at a first hinge region, and ii) a central part forming a second valve flap secured to the first valve flap at a second hinge region, said first and second valve flaps collaborating with one another in a completely hermetic manner.

The invention will be better understood in the light of the following examples and the following figures, in which the bracketed references relate to the figures appended to the present description.

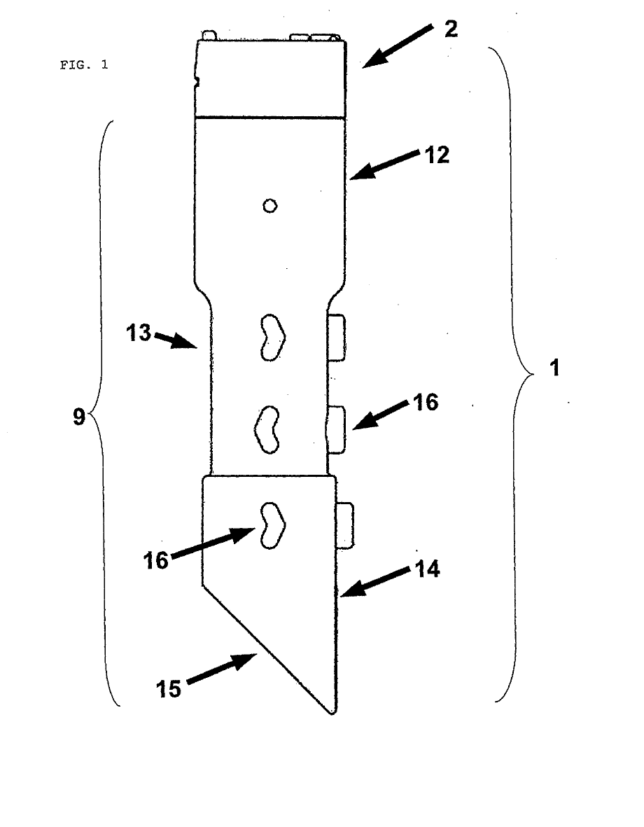

FIG. 1 describes a profile view of a device (1) forming an intralaryngeal prosthesis for a dysfunctional larynx according to the invention. It shows the annular bearing structure (2) the upper part of which is equipped with valve flaps forming a shutter and the lower part which is equipped with a silicone skirt (9), intended to be fitted into the larynx, at the top of the trachea. It also depicts the various parts that make up the skirt (9), namely the first part (12) fixed to the annular bearing structure, the second part (13) and the third part (14) which is in the shape of a bevel (15). Lugs (16) aimed at blocking the prosthesis once it has been implanted can be seen on the exterior part of the skirt (9).

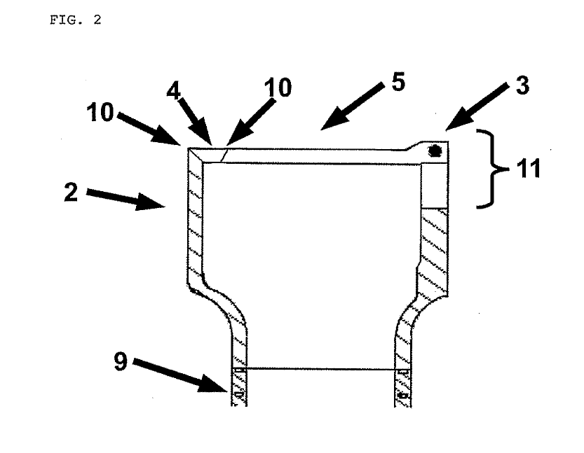

FIG. 2 is a view in vertical section of the upper part of a device (1) forming an intralaryngeal prosthesis for a dysfunctional larynx according to the invention. It shows the annular bearing structure (2) in the upper part of the device (1) and the collar or skirt (9) fixed to the lower part of the annular bearing structure (2). The large valve flap (4) and the small valve flap (5) are borne by the annular bearing structure (2) and are articulated about a hinge zone (3). In this embodiment, the hinge zone (3) is a rigid pin which fits into a cutout made in the valve flaps (4) and (5). Limit stop elements (10) allow the valve flaps to be blocked depending on the embodiment chosen. The valve flaps (4) and (5) in the closed position form a shutter (11).

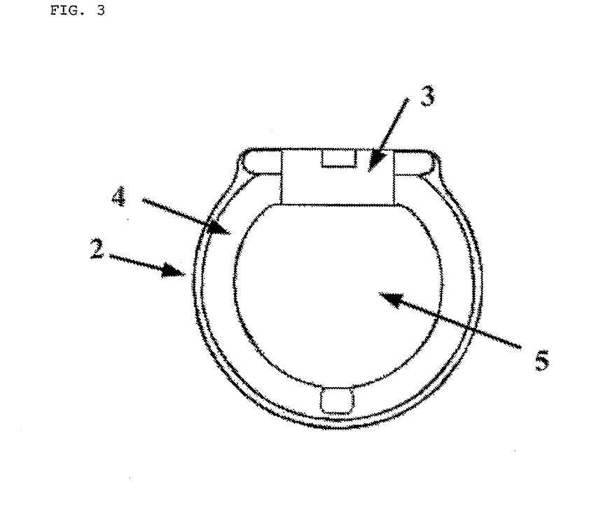

FIG. 3 is a view from above of the device (1) which forms an intralaryngeal prosthesis according to the invention. It shows the large valve flap (4) and the small valve flap (5) and the circumference of the annular bearing structure (2). It also shows the hinge zone (3) which corresponds to a model like the one described in FIG. 2, namely a hinge with a rigid pin.

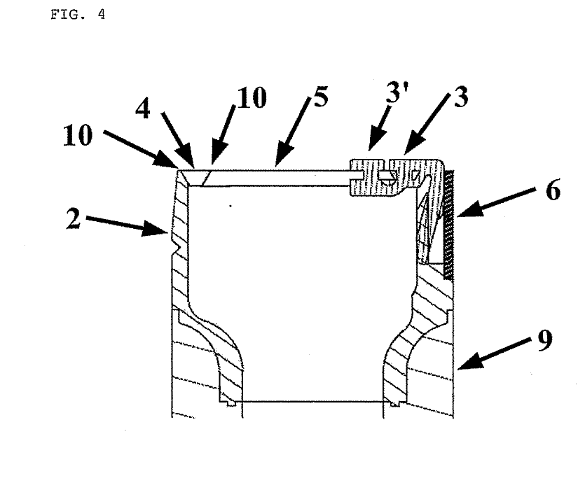

FIG. 4 describes a view in vertical section of the upper part of the device (1) that forms an intralaryngeal prosthesis for a dysfunctional larynx according to the invention. It illustrates the shutter formed by the valve flaps (4, 5) in the horizontal position, the limit stops (10), a first hinge zone (3), a second hinge zone (3′), a fixing plate (6) and the annular bearing structure (2) and the collar or skirt (9). This model details a hinge zone made from a semi-rigid material which takes the form of a tab fixed to the valve flaps (4) and (5) at one of its ends and blocked by the plate (6) at its other end. The valve flaps (4) and (5) in the closed position form a shutter (11).

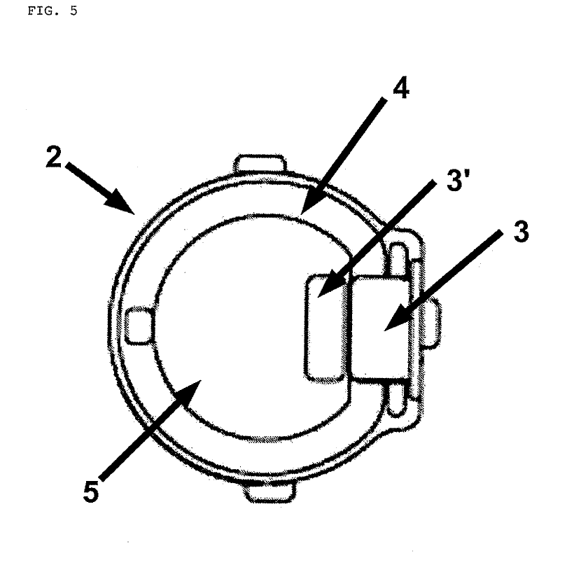

FIG. 5 is a view from above of a device (1) forming an intralaryngeal prosthesis according to the invention. It shows the large valve flap (4) and the small valve flap (5) and the circumference of the annular bearing structure (2). It also shows a first hinge zone (3) and a second hinge zone (3′) which corresponds to a model like the one described in FIG. 4, namely a semi-rigid hinge.

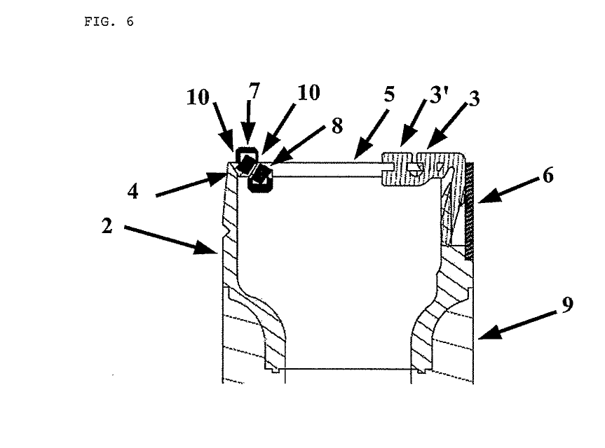

FIG. 6 describes a view in vertical section of the upper part of the device (1) that forms an intralaryngeal prosthesis for a dysfunctional larynx according to the invention. This is the model described in FIGS. 4 and 5, namely a model with a semi-rigid hinge. It illustrates the shutter formed by the valve flaps (4) and (5) in a horizontal position, the limit stops (10), the hinge zones (3) and (3′), the assistance elements (7, 8), the fixing plate (6) and the annular bearing structure (2) and the collar or skirt (9). The assistance elements encourage the valve flaps to close.

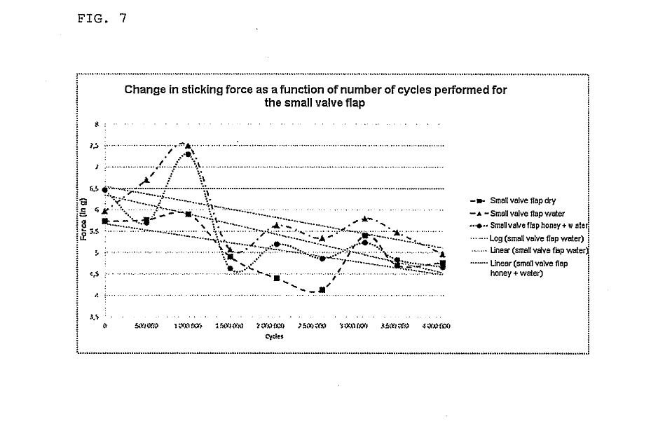

FIG. 7 is a graph describing how the sticking forces change as a function of the number of breathing cycles performed in the case of the small valve flap (5).

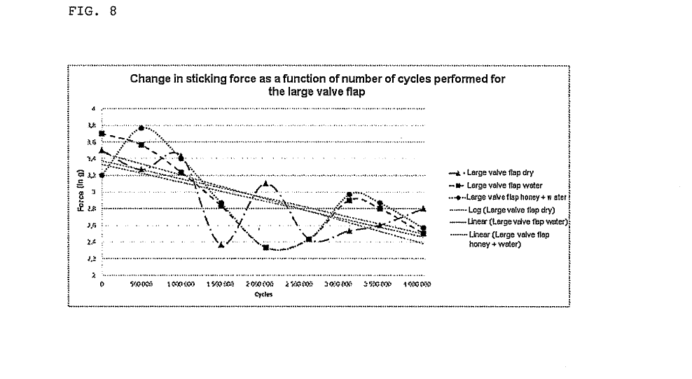

FIG. 8 is a graph describing how the sticking forces change as a function of the number of breathing cycles performed in the case of the large valve flap (4).

The device according to the invention is made up predominantly of biocompatible solid metal such as titanium or titanium-based alloys, gold or gold-based alloys, and platinum or platinum-based alloys, niobium and tantalum or alloys of these or more generally of any other biocompatible solid metal.

In a preferred embodiment, the metal used to manufacture the annular bearing structure (2) and the valve flaps (4) and (5) is titanium or a titanium-based alloy. This is because this material is perfectly biocompatible and it is possible to manufacture components of small size the thickness of which can easily be mastered. The present invention describes a flap valve device (1) intended to be implanted in a dysfunctional larynx, by means of a silicone skirt (9), said device comprising a distal portion forming an annular bearing structure (2) and a central portion forming a shutter (11) intended to allow air to pass and to hermetically prevent any other element from passing, the shutter (11) comprises i) a peripheral part forming a first valve flap (4) secured to the annular bearing structure (2) at a first hinge region (3), and ii) a central part forming a second valve flap (5) secured to the first valve flap at a second hinge region (3′), and a means forming a collar or skirt (9) for fixing the intralaryngeal prosthesis into, and removing it from, a dysfunctional larynx, situated below the annular bearing structure (2), in which the annular bearing structure (2) and the two valve flaps (4) and (5) that form the shutter (11) are made of solid metal, preferably titanium or a titanium-based alloy.

The method of manufacturing the device according to the present invention entails microtechnical skills. This is because in order to ensure that the device according to the invention and more particularly the shutter seals perfectly, the various parts made of solid metal need to be perfectly matched and the operation of the valve flaps needs to be extremely precise and reliable in terms of the repeatability of the movements. To achieve that, each component is produced with particular care before being assembled with the other parts that make up the device according to the invention. The hinge zone adopts an especial importance insofar as its role is to allow the valve flaps to fall and to rise in order to restore the functions of the dysfunctional larynx in which the device is inserted. This role is repetitive and of high precision. This is because, without a reliable hinge function, the device would remain blocked either in the open position (with no protection of the lower airways) or in the closed position (without the possibility of air circulating), or the valve flaps would be misaligned leading to an absence of sealing when closed, leading to complications that are serious for the patient. In the present invention, the hinge zone needs to allow valve flaps made of solid metal to open and needs to return these to the closed position using a return force that it applies. The hinge zone is therefore an articulation zone which is either linked with the mechanical properties of the material used or linked with the operation of a mechanism that allows the movement.

In the present description, the expressions “valve flap”, “valve”, “shutter” or even “flap valve” can be used interchangeably to denote a moving part capable of moving from a closed position, or shut-off position, to an open position that allows air to pass.

The expressions “distal” and “central” have as their frame of reference the center of the device according to the invention. It therefore follows that the expression “distal portion” refers to the portion furthest from the center of the device as opposed to the expression “central portion” which refers to the portion closest to the center of said device according to the invention.

The terms “semi-rigid” and “superelastic” are used with reference to soft materials that have spontaneous and permanent rigidity properties or rigid materials that have spontaneous and permanent elasticity properties. In general, these materials permit a certain elasticity without permanent deformation and therefore spontaneously return to their initial shape. By way of example of semi-rigid materials, mention may be made of plastics, rubber, silicone, and by way of example of superelastic materials, mention may be made of nitinol (a nickel-titanium alloy).

In a preferred embodiment, the circular structure of the two valve flaps is preferable because, for a given size, it is this configuration that offers the widest possible opening for the passage of the air. However, such a shape is not in any way limiting and the device according to the invention may exhibit other shapes or structures, notably an ovoid cross section.

At rest, the valve flaps of the device according to the invention nestle one inside the other in the same plane. However, depending on the mode of collaboration chosen, the set of two valve flaps falls during inhalation whereas only the smaller valve flap rises during exhalation, or alternatively the set of the two valve flaps rises during exhalation whereas only the smaller valve flap falls during inhalation.

Among the possible modes of embodiment, the two valve flaps may have their travel blocked by a limit stop (10) or by a system of magnetic type. It is also conceivable to combine limit stop elements (10) with a system of magnetic type.

Another way of explaining this is to state that the invention relies on the collaboration of two valve flaps (4) and (5) nestling one inside the other in the same plane at rest, the set of two valve flaps falling on inhalation whereas only the smaller one opens on exhalation or alternatively the set of two valve flaps opening on exhalation whereas only the central valve flap falls on inhalation. When the two valve flaps (4) and (5) are in the same plane at rest, they form a shutter (11) which is hermetic.

The valve flaps (4) and (5) are made to work by the raised pressure and reduced pressure in the lungs during the exhalation and inhalation phases. In one of the embodiments, during inhalation and because of the reduced pressure prevailing in the lungs, the two valve flaps (4) and (5) fall in the same movement. On exhalation, because of the raised pressure prevailing in the lungs only the small valve flap (5) opens toward the outside of the device, the large valve flap (4) remaining blocked on its seat. In another embodiment of the present invention, during inhalation and because of the reduced pressure prevailing in the lungs, the central small valve flap (5) falls by itself, the large valve flap (4) remaining blocked on its support. On exhalation, because of the raised pressure prevailing in the lungs, the two valve flaps (4) and (5) rise toward the outside of the device in the same movement. The guarantee of good fluidtightness of the trachea upon closure of the valve flaps (4) and (5), combined with a good mechanical strength in the device according to the invention is a very important factor during swallowing in order to prevent food or saliva from entering the trachea and then the lungs.

In one particular embodiment, the valve flap (4) is capable of falling under the effect of the reduced pressure resulting from the patient inhaling, and the valve flap (5) is capable i) of collaborating with the valve flap (4) by falling but also ii) of rising under the effect of the raised pressure exerted by the air exhaled by the patient. In one particular embodiment of the device (1) according to the invention, the internal part of the valve flap (4) comprises at least one limit stop element (10) preventing the valve flap (5) from rising.

In another particular embodiment, the valve flap (4) is capable of rising under the effect of the raised pressure applied by the air exhaled by the patient and the valve flap (5) is capable i) of collaborating with the valve flap (4) by rising but also ii) of falling under the effect of the reduced pressure resulting from the patient inhaling. In one particular embodiment of the device (1) according to the invention, the internal part of the annular bearing structure (2) comprises at least one limit stop element (10) preventing the valve flap (4) from falling.

In one preferred embodiment of the device (1) according to the invention, the limit stop element (10) consists of the fact that the external circumference of the first valve flap (4) of the shutter (11) is cut with a “downward” bevel, and that the internal circumference of the annular bearing structure (2) facing it is also cut with an “inverse upward” bevel, the two bevel cuts collaborating with one another to block the rise of the first valve flap (4).

As will become apparent from the examples below, one of the features of the invention relies on the material used from which to manufacture the annular bearing structure (2) and the valve flaps (4) and (5) of the device (1) according to the invention. This assembly is also referred to as the active part of the device. The thickness of the valve flaps (4) and (5), their system of articulation and their overall shape allowing perfect sealing are also decisive factors in optimum operation of the device (1). This is because since the preferred material is solid titanium, the valve flaps (4) and (5) have no elasticity unlike those described in the prior art. The role of the hinge regions (3) and (3′) is therefore key to ensuring even and sufficient movement according to the demands made by the patient. Specifically, the hinge zone (3), whatever its configuration according to the present invention, has a direct influence on the ability of the device (1) to fall or even to hold its position. This elasticity needs to be evaluated so that the pressure corresponding to the thrust applied by food or by a buildup of saliva, mucus or any other fluid can be resisted to prevent it from entering the upper airways, while at the same time the valve flap is capable of falling or rising in response to an exhalation raised pressure or to an inhalation reduced pressure.

In one preferred embodiment, the hinge regions (3) and (3′) are situated between the valve flaps (4) and (5) and the annular bearing structure (2) to form just one single hinge zone (3, 3′) for the two valve flaps (4) and (5). Finally, in one particular embodiment, the hinge regions (3) and (3′) are made of a semi-rigid, superelastic or rigid material. In the case of a hinge zone made of a rigid material, this is, for example, a metallic pin around which the corresponding valve flap is articulated by means provided for that purpose during the cutting-out of the component (see FIG. 2). In the case of a hinge zone produced in a semi-rigid or superelastic material, this is, for example, silicone (see FIG. 4) or nitinol.

In the remainder of the description, the expression “alimentary bolus” will be used to define not only any element of said alimentary bolus but also mucus, saliva or any other element or body that is foreign to the upper and lower airways.

According to one preferred embodiment of the invention, the device is characterized in that the annular bearing structure (2) comprises at least one limit stop element (10) preventing the first valve flap (4) from falling. This limit stop element (10) performs a key role because it makes it possible either to prevent any movement of the first valve flap (4) downward following the inhalation of the patient, or to prevent any movement of the first valve flap (4) upward following exhalation by the patent.

Said limit stop element (10) may consist of any means that impedes and blocks the upward or downward movement of the first valve flap (4), such as a lug projecting over the entire internal circumference of the annular bearing structure for example, or alternatively simply arranged at one or more precise points on this circumference.

In one particular embodiment, the limit stop element (10) intended to block the movement of the valve flap (4) either downward or upward depending on the device chosen is a particular cut of the external circumference of the first valve flap (4) of the shutter (11) and an inverse cut of the internal circumference of the annular bearing structure (2) in its top part. The two cuts are intended to collaborate in such a way as to hermetically block the rising of the valve flap (4). By way of example of such a limit stop system that uses particular cuts, mention will be made of the device made of a semi-rigid material such as silicone described in European patent application EP 08872036. The reverse operation to that described in EP 08872036 may also be envisioned in the present invention, namely for the valve flaps (4) and (5) to collaborate to rise when the patient exhales. The valve flap (5) will fall during inhalation. In the case of such a system with a cut forming a limit stop (10), a bevel cut is preferred because it allows a better seal to be afforded. However, any other shape that allows such collaboration must of course also be considered to be equivalent.

Such bevel cuts or cuts in any other equivalent shape are perfectly feasible in a rigid material such as titanium. This limit stop system (10) has the advantage of being simple to implement, and avoids adding additional elements to the device (1) according to the invention, and notably to the active part of said device.

As described in European patent application EP 08872036, the cuts described hereinabove for limiting the movement of the valve flap (4) with respect to the upper part of the annular bearing structure (2) can also be used to join the valve flaps (4) and (5) together. The cuts will then be made on the interior circumference of the valve flap (4) and on the exterior circumference of the valve flap (5). Depending on the embodiment chosen, namely whether the two valve flaps (4) and (5) fall together or whether the two valve flaps (4) and (5) rise together, the directions of cut will be chosen to be “upward” or “downward”.

As detailed hereinabove, the two valve flaps (4) and (5) that form the shutter (11) are actuated only by the effects of reduced pressure and raised pressure caused by the patient's breathing and, on the other hand, need to be immobile as a result of some other pressure such as the weight of any foreign element that could, for example, come from the alimentary bolus. In order to meet this requirement, the thickness of the shutter (11) is between 0.3 mm and 2.0 mm. In one particular embodiment, the shutter (11) has a thickness of 1.0 mm to 1.5 mm. In a preferred embodiment, the shutter (11) has a thickness of 1.0 mm.

By comparison with the valves and/or valve flaps described in the prior art and generally made of a soft material such as silicone, the valve flaps (4) and (5) according to the invention are made of solid titanium or a titanium-based alloy and are fixed by a hinge region to an annular bearing structure (2) likewise made of solid titanium or a titanium-based alloy. These valve flaps are therefore particularly able to resist the arrival of fluids or of the alimentary bolus at the device, while at the same time beginning to move with the patient's, breathing.

The weight of the alimentary bolus is estimated on average at 7.0 g which translates to a pressure which, on average, does not exceed 3.10⁻³ MPa. The alimentary bolus is made up of partially or fully chewed food impregnated with saliva. After it has been swallowed, this alimentary bolus will travel along the esophagus before finally reaching the stomach. Its mass will break up to some extent following chewing and swallowing and according to the nature and weight of the foodstuffs of which it is made, and the swiftness of its path along the esophagus will similarly vary. Under physiological conditions, it may therefore be reasonably considered that not all of said alimentary bolus will reach a specific point of the device according to the invention at the same time. The flap valve device according to the invention is designed to resist an average load of 7.0 g, and this is easily enough for correct operation of the flap valve device.

Moreover, the raised pressure applied when the patient exhales and the reduced pressure applied when he inhales are of the order of 10⁻² MPa, namely in both instances far greater than the average pressure applied by the alimentary bolus, around 3.10⁻³ MPa. It will therefore be easily understood that the valve flaps (4) and (5) can rise or fall without difficulty as the patient breathes.

More specifically, the device according to the invention is characterized in that the first valve flap (4) or the second valve flap (5), depending on the way in which the device works, and their hinges are dimensioned so as to be able to withstand a load less than 4.0 g without rising and/or falling, depending on the embodiment of the invention chosen.

In an alternative form, it may be desirable for the resistances of the two valve flaps (4) and (5) to differ so as to be able for example to adapt to suit patients presenting respiratory problems that lead to differences in pressure between inhalation and exhalation. In this specific case, the dimensions of the two valve flaps (4) and (5), and of the corresponding two hinge regions (3) and (3′), may differ.

In general, it is preferable for the first valve flap (4) and the second valve flap (5) and the first (3) and second (3′) hinge regions to have the same thickness. This avoids there being protruding zones which could cause fluid or undesirable residue to build up. Moreover, this last form of embodiment has numerous advantages, for example reduction in manufacturing costs.

In a preferred embodiment according to the invention, in order to increase the resistance to the pressure applied by fluids while not preventing the operation of the valve flaps (4) and (5), an assistance device, which may be mechanical, electrical or electronic, is used. Said assistance device (7, 8) is placed at the annular bearing structure (2) and/or at the first valve flap (4). The present invention also relates to a flap valve device (1) for an intralaryngeal prosthesis of which the annular bearing structure (2) and/or the first valve flap (4) are provided with an assistance device (7, 8) that can be mechanical, electrical or electronic.

In one particular embodiment, the assistance device (7, 8) described hereinabove is a magnetized device. What is meant by a magnetized device or a magnetized element is one or more permanent magnets of the lanthanide type which are biocompatible or rendered biocompatible by various treatments known to those skilled in the art, or alternatively hermetically sealed in a suitable housing provided for that purpose. Said magnetized device may be positioned either on the first valve flap or on the internal surface of the bearing structure of the device according to the invention. Placed facing the magnet or magnets when the first valve flap is in the closed position, is a metallic element. What is meant by metallic element is one or more metallic elements that can be magnetized which are placed in such a way as to come into contact with the magnet or magnets when the first valve flap is in the closed position. The nature of the magnetized device may be adapted to suit each situation by varying the number of magnets and/or their position for example. It may also be conceivable to use two magnets facing one another.

In one particular embodiment, the present invention relates to a flap valve device for an intralaryngeal prosthesis (1) comprising an assistance device (7, 8) which consists of a metallic element positioned at the annular bearing structure (2) coming into contact with a magnetized element arranged at the first valve flap (4).

In one preferred embodiment, the present invention relates to a flap valve device for an intralaryngeal prosthesis (1) comprising an assistance device (7, 8) which consists of a metallic element positioned at the first valve flap (4) coming into contact with a magnetized element positioned at the annular bearing structure (2).

As described hereinabove, a mechanical assistance device can be positioned on each of the valve flaps to make it easier for them to cooperate in the desired operation. According to this embodiment, the present invention describes a device in which the first valve flap (4) and the second valve flap (5) comprise an assistance device so as to allow the rising of only the second valve flap (5) and the simultaneous falling of the first valve flap (4) and of the second valve flap (5). In another embodiment, the valve flaps (4, 5) of the device according to the invention comprise an assistance device so as to allow the falling of only the second valve flap (5) and the simultaneous rising of the first valve flap (4) and of the second valve flap (5).

The magnetized device described hereinabove has a function of mechanically assisting the flap valve device (1) according to the invention. More generally, it will be easy for a person skilled in the art to replace or supplement this magnetized device with any device that performs an equivalent function or is capable of improving this function, such as another assistance device that may be mechanical, electrical or electronic.

Furthermore, it is possible for the hinge regions (3) and (3′) and the valve flaps (4) and (5) not to have the same thicknesses, it being possible for these thicknesses to be adapted to suit a specific situation dictated by the actual condition of the patient.

In order to be able to ensure the movement of the valve flap or flaps while at the same time maintaining perfect sealing, the hinges (3) and (3′) need to be made from a semi-rigid or even a rigid material. Optimizing hinge regions made from a semi-rigid or rigid material requires a very special approach because it is necessary both for movement to be rendered possible with a low respiratory pressure and also for the hinges to allow the valve flaps to resist the pressure of the alimentary bolus or of any fluid that might press against the shutter with a varying degree of force. Moreover, as stated hereinabove, it is necessary for the hinges to resist colonization and to provide smooth and lasting movement of the valve flaps. In addition, the hinge zone needs to ensure perfect stability of the rotation pins with respect to the bearing structure of the device so as to guarantee sealing on closure. The present invention proposes several types of hinge capable of meeting all these mechanical requirements. The principles of operation when choosing a semi-rigid material, a superelastic material or a rigid material will not be the same.

In the first instance, a semi-rigid or a superelastic material means in the present invention a material that is soft enough to be able to provide a certain degree of elasticity while at the same time being rigid enough to be able to resist and remain in shape under the effect of a small pressure. Such a semi-rigid material may be selected from plastics, rubber, a resin or even silicone. A superelastic material such as nitinol may also be envisioned. A preferred material is 70 Shore A silicone. The hinge then consists of a tab of silicone fixed to the exterior part of the annular bearing structure (2) by two studs and of a solid titanium plate (6). The other end of the semi-rigid tab is fixed to the valve flaps (4) and (5) by any suitable means. By way of example, mention will be made of the perforating of a small part of the valve flap, creating orifices into which the semi-rigid material will flow. This mechanism has the benefit of being perfectly functional and biocompatible. In one particular embodiment, the present invention describes a flap valve device (1) comprising a distal portion forming an annular bearing structure (2) and a central portion forming a shutter (11), the shutter (11) comprises i) a peripheral part forming a first valve flap (4) secured to the annular bearing structure (2) at a first hinge region (3), and ii) a central part forming a second valve flap (5) secured to the first valve flap at a second hinge region (3′), and a means forming a collar or skirt (9) for fixing the intralaryngeal prosthesis into, and removing it from, the dysfunctional larynx situated below the annular bearing structure (2), characterized in that the annular bearing structure (2) and the two valve flaps (4) and (5) that form the shutter (11) are made of solid metal, preferably made of titanium or a titanium-based alloy and that the hinge regions (3, 3′) are made of one or more semi-rigid or superelastic materials.

In the second instance, the hinge will be made from a rigid material. A rigid material means a mechanism such as a mechanical hinge, consisting of a rigid pin which fits into a cut made in the valve flaps (4) and (5). The rigid pin may be made of ruby or from a metal such as titanium or a titanium-based alloy or any other rigid biocompatible metal. More generally, all the mechanisms used in horology and made from biocompatible materials can be used in implementing the present invention. This mechanism has the benefit of being robust because it is made of materials that are rigid and biocompatible which deteriorates little if at all. In one preferred embodiment, the present invention describes a flap valve device (1) comprising a distal portion forming an annular bearing structure (2) and a central portion forming a shutter (11), the shutter (11) comprises i) a peripheral part forming a first valve flap (4) secured to the annular bearing structure (2) at a first hinge region (3), and ii) a central part forming a second valve flap (5) secured to the first valve flap at a second hinge region (3′), and a means forming a collar or skirt (9) for fixing the intralaryngeal prosthesis into, and removing it from, the dysfunctional larynx situated below the annular bearing structure (2), characterized in that the annular bearing structure (2) and the two valve flaps (4) and (5) that form the shutter (11) are made of solid metal, preferably of titanium or titanium-based alloy and that the hinge regions (3, 3′) are made of one or more rigid materials.

The means forming a collar or skirt (9) which is situated below the annular bearing structure (2) has the role of allowing the intralaryngeal prosthesis to be placed and held within a dysfunctional larynx. This skirt (9) is made up of three parts. The first part (12) is situated at the top of the device (1) and fixed to the annular bearing structure (2) using holes that allow optimal bonding with the material of which said skirt (9) is made, the second part (13) of a smaller diameter than the first part (12) is situated below the latter, and finally the third part (14) of a diameter greater than that of the part (13) is situated below the second part (13) and comprises a bevel or cone-frustum cut (15). The variations in diameter between the three parts make it possible to ensure that the prosthesis once implanted remains in an axial position because this allows it to adapt to suit the geometry of the patient's anatomy.

The skirt (9) can be made of any biocompatible semi-rigid material. It can be made of a material which is solid or in the form of a spring or “stent”. In one preferred embodiment, the skirt (9) is made of silicone. This is because since silicone is deformable it allows the skirt to penetrate the orifice of the larynx, of the cricoid cartilage and the vocal cords and thus position the third part (14) at the top of the trachea, the cricoid cartilage and the vocal cords being situated level with the second part (13) and the first part (12) lying above the previous two. The upper plane of the prosthesis is then situated approximately at a distance of between 10 mm and 15 mm above the plane of the arytenoid cartilage. The bevel (15) or cone frustum makes it easier for the entire device (1) according to the invention to enter the patient's larynx.

The skirt (9) made of silicone is also provided with lugs (16) intended to block the prosthesis against translational and rotational movement once it is in position within the patient's larynx. The inside diameter of the skirt (9) allows the passage of a 6.0 mm diameter cannula so that medical examinations can be performed if required. Finally, the inside of the skirt (9) has a planar additional thickness over the entire height of the device (1) according to the invention so as to increase the rigidity of the device (1) while it is being implanted. Most of the sharp corners of the skirt (9) are rounded to make them non-traumatic on the one hand, and to make removal of mucosities from the lower airways to the pharyngeal zone easier on the other.

### EXAMPLES

Various tests have been conducted on two intralaryngeal prostheses for a dysfunctional larynx according to the invention in order to ensure that their mechanical properties meet the needs of the patients. These tests consist of a fatigue test on the articulation or hinge zone (3, 3′) of the device according to the invention, of a leak test testing the sealing of the valve flaps against liquids and of a shutter (11) sticking test. The model of flap valve device according to the invention tested here has the following characteristics:
the hinge regions (3) and (3′) are situated on the same side of the device;
the articulation or hinge region (3, 3′) is made of silicone.

### 1. Articulation Fatigue Test

The valve flaps (4) and (5) are secured to a silicone articulation (3, 3′) fixed to the titanium annular bearing structure (2) of the flap valve device that forms the intralaryngeal prosthesis (1). This articulation (3, 3′) allows the valve flaps (4) and (5) on the one hand to be at rest in the closed position and on the other to allow the small valve flap (5) to open downward, during inhalation, and allow the large valve flap (4) and the small valve flap (5) to open simultaneously upward during exhalation.

In a patient at rest, the rate of breathing is on average 12 times per minute, namely 720 times per hour and 17 280 times per day or 518 400 per month. If an implantation life of at least 3 months in patients is considered, with a factor of safety of 2, that represents 3 110 400 breathing cycles and therefore loading cycles on the silicone articulation (3, 3′).

In order to make sure of the factor of safety, the two devices according to the invention tested were subjected to a pneumatic test opening and closing the valve flaps (4) and (5) at a frequency of 2 Hz and sometimes of 1 Hz for a duration in excess of 4 million cycles. Table 1 below shows the results of the test for one of the devices according to the invention, with detailed observations under a binocular on each stoppage.

TABLE 1
articulation fatigue test

| | | | Duration | | Number of |
|---|---|---|---|---|---|
| | Start of phase | End of phase | of phase | Frequency | cycles |
| Step | (date and time) | (date and time) | (h:min:s) | (Hz) | performed |
| 1 | 15 Dec. 2009 19:06 | 16 Dec. 2009 14:52 | 19:48:00 | 2 | 142 320 |
| 2 | 16 Dec. 2009 15:28 | 17 Dec. 2009 18:08 | 02:40:00 | 2 | 334 320 |
| 3 | 17 Dec. 2009 19:01 | 18 Dec. 2009 18:31 | 23:30:00 | 2 | 503 520 |
| 4 | 18 Dec. 2009 20:39 | 21 Dec. 2009 18:53 | 22:14:00 | 2 | 1 009 200 |
| 5 | 22 Dec. 2009 19:02 | 28 Dec. 2009 15:43 | 20:41:00 | 1 | 1 515 660 |
| 6 | 30 Dec. 2009 19:44 | 4 Jan. 2010 9:48 | 14:04:00 | 1 | 1 911 900 |
| 7 | 4 Jan. 2010 10:18 | 5 Jan. 2010 9:40 | 23:22:00 | 2 | 2 080 140 |
| 8 | 5 Jan. 2010 11:06 | 8 Jan. 2010 15:05 | 03:59:00 | 2 | 2 827 220 |
| 9 | 8 Jan. 2010 16:22 | 11 Jan. 2010 15:46 | 23:24:00 | 2 | 3 141 300 |
| 10 | 11 Jan. 2010 17:06 | 13 Jan. 2010 14:20 | 21:14:00 | 2 | 3 466 980 |
| 11 | 13 Jan. 2010 14:20 | 14 Jan. 2010 8:33 | 18:13:00 | 1 | 3 532 560 |
| 12 | 15 Jan. 2010 12:22 | 18 Jan. 2010 14:45 | 02:23:00 | 2 | 4 068 120 |
| 13 | 18 Jan. 2010 16:44 | 18 Jan. 2010 17:47 | 01:03:00 | 2 | 4 075 680 |
| 14 | 19 Jan. 2010 9:35 | 19 Jan. 2010 10:20 | 00:45:00 | 2 | 4 081 080 |

It will be noted that after more than 4 million cycles, the two devices of the invention are intact, with no beginnings of cracks in the silicone articulation (3, 3′). The parts made of titanium show no alteration. The two devices are moreover perfectly functional.

The only observations made are that a small amount of peening occurs in the zones of contact between the valve flaps and the annular bearing structure. However, this has no impact on the operation of the device according to the invention.

### 2. Valve Flap Leak Test for Sealing Against Liquids

As described in the description, the device according to the invention is placed in the larynx. It is therefore subjected to the passage of the alimentary bolus and notably to the liquids which may be water or liquids of various kinds.

This test is performed using a blotting paper. The latter is weighed beforehand then placed under the valve flaps (4) and (5). A certain quantity of water is then poured over the valve flaps (4) and (5) kept in the closed position and therefore forming a shutter (11). The results are detailed hereinbelow in table 2.

In order to define an acceptance criterion that is compatible with what is acceptable to the trachea under physiological conditions, it was decided that the increase in mass of the blotting paper has to remain below 1% of the mass of water poured, corresponding to the contents of 5 soupspoons, poured in succession, namely around 62 grams of water.

This test was performed at each stoppage for observing the condition of the articulation or hinge zone (3, 3′) and that of the valve flaps (4) and (5) during the fatigue tests.

TABLE 2
Leak test for seal against liquids

| | Measured | | Corresponding | | | |
|---|---|---|---|---|---|---|
| Number of | soaked | | mass of | | | |
| cycles | length (cm) | | water (g) | | % leaks | % leaks |
| performed | L1 | L2 | M1 | M2 | 1 | 2 |
| 0 | — | — | — | — | — | — |
| 503 520 | — | — | — | — | — | — |
| 1 009 200 | — | — | — | — | — | — |
| 1 515 660 | 5 | 4 | 0.10 | 0.06 | 0.16 | 0.13 |
| 2 080 140 | 4.5 | 6.5 | 0.09 | 0.13 | 0.15 | 0.21 |
| 2 827 220 | 7 | 7 | 0.14 | 0.14 | 0.23 | 0.23 |
| 3 141 300 | 6 | 6 | 0.12 | 0.12 | 0.20 | 0.20 |
| 3 532 560 | 7 | 8 | 0.14 | 0.16 | 0.23 | 0.26 |
| 4 081 080 | 8.5 | 7.5 | 0.17 | 0.15 | 0.28 | 0.25 |

The first leaks are measured after 1 500 000 breathing cycles. The leakage percentages are very small but do increase with the number of cycles performed.

The measured leaks are still at very low levels, representing less than 0.28% for one of the devices and less than 0.26% for the other device. That demonstrates the good sealing of the two valve flaps (4) and (5) forming a shutter (11) in each of the devices tested.

### 3. Sticking Test

These tests are aimed at assessing the effect of certain viscous products that the patient ingests on the force necessary to open the two valve flaps (4) and (5) during a breathing cycle. These tests were carried out using a mixture of 50% honey and 50% water likenable to the viscosity of a product made up of honey and saliva during the swallowing phase. The measurements were performed using a dynamometer during each stoppage of the fatigue tests for checks. FIGS. 7 and 8, which relate to the small valve flap (5) and to the large valve flap (4), show that the opening force does not vary appreciably over the life of the implant.

FIG. 7 shows that the opening force for the small valve flap (5) does not vary appreciably over the life of the device according to the invention.

FIG. 8 shows that the opening force for the large valve flap (4) does not vary appreciably over the life of the device according to the invention.

### 4. Cough Test

These tests are aimed at checking that the devices according to the invention, under the effect of a cough reflex, will open properly to allow the expelled air out and that they will return to their rest position, forming the shutter (11), without deformation so as to prevent the alimentary bolus from entering the trachea. This test was carried out by performing 100 impulses corresponding to coughing during each of the stoppages for checking of the devices during the fatigue tests. The results showed that the implants maintained their functionality and their integrity on completion of these tests.

In general, the present description is aimed at illustrating the invention in the clearest possible way and any obvious embodiment modification must be considered as being equivalent and, therefore, must be considered as being covered by the claims that follow which define the scope for which protection is sought.

1. A flap valve device (1) intended to be implanted in a dysfunctional larynx, said device comprising
an annular bearing structure (2),
a shutter (11) configured to allow air to pass and to hermetically prevent any other element from passing, wherein the shutter (11) comprises i) a first valve flap (4) secured to the annular bearing structure (2) at a first hinge region (3), and ii) a second valve flap (5) secured to the first valve flap at a second hinge region (3′), and
a collar or skirt (9) secured to the annular bearing structure (2) for fixing the intralaryngeal within the dysfunctional larynx,
wherein the annular bearing structure (2) and the two valve flaps (4) and (5) that form the shutter (11) are made of a solid metal.

2.-14. (canceled)

15. The device as claimed in claim 1, wherein the solid metal comprises titanium or of a titanium-based alloy.

16. The device as claim in claim 1, wherein the hinge regions (3) and (3′) are situated between the valve flaps (4) and (5) and the annular bearing structure 92) to form a single hinge zone (3, 3′) for the two valve flaps (4) and (5).

17. The device as claimed in claim 1, wherein the hinge regions (3) and (3′) comprise a semi-rigid, superelastic or rigid material.

18. The device as claimed in claim 17, wherein the hinge regions (3) and (3′) consist of a silicone tab blocked by a titanium plate 96) wherein orifices made in the surface of the valve flaps (4) and (5) allow the entry of the silicone to firmly anchor the tab.

19. The device as claimed in claim 17, wherein the hinge regions (3) and (3′) consist of a rigid pin on which the two valve flaps (4) and (5) are articulated.

20. The device as claimed in claim 19, wherein the pin comprises a material selected from ruby or a metal.

21. The device as claimed in claim 1, wherein an internal part of the valve flap (4) comprises at least one limit stop element (10) preventing the valve flap (5) from rising.

22. The device as claimed in claim 21, wherein an internal part of the annular bearing structure (2) comprises at least one limit stop element (10) preventing the valve flap (4) from falling.

23. The device as claimed in claim 22, wherein the limit stop element (10) on the first valve flap (4) comprises a “downward” bevel, and the limit stop elements on the annular bearing structure (2).

24. The device as claimed in claim 1, further comprising an assistance device configured to allow only the second valve flap (5) to rise and the first valve flap (4) to fall together with the second valve flap (5).

25. The device as claimed in claim 1, further comprising an assistance device configured to allow only the second valve flap (5) and the first valve flap (4) to rise together with the second valve flap (5).

26. The device as claimed in claim 1, wherein the collar or skirt (9) comprises silicone.

27. The device as claimed in claim 26, wherein the skirt (9) comprises a first part (12) situated at the top of the device (1) and fixed to the annular bearing structure (2) using holes that allow bonding with the silicone, a second part (13) of a smaller diameter than the first part (12) situated below the first part and a third part (14) of a diameter greater than that of the second part (13) and situated below the second part (13) and which comprises a bevel cut (15), the exterior surface of the skirt (9) being provided with lugs (16) and the interior surface of the skirt (9) having a planar additional thickness over its entire height.
